(19) United States
(12) Patent Application Publication
(10) Pub. No.: US 2024/0087717 A1
(43) Pub. Date: Mar. 14, 2024

(54) METHODOLOGY AND APPLICATION OF MAKING CUSTOMIZED COMPOSITIONS OF SUPPLEMENTS

(71) Applicants: Meesue Kim, Kirkland, WA (US); Yoo Seung Kim, Bangkok (TH)

(72) Inventors: Meesue Kim, Kirkland, WA (US); Yoo Seung Kim, Bangkok (TH)

(21) Appl. No.: 17/931,126

(22) Filed: Sep. 11, 2022

(51) Int. Cl. G16H 20/60; G16H 20/90; G16H 40/67

(52) U.S. Cl. CPC G16H 20/60; G16H 20/90; G16H 40/67

(57) ABSTRACT

The present invention describes the methods of applying health-related data that may be generated through IoT devices. This data is analyzed through artificial intelligence algorithms based on various types of knowledge (Chinese medicine, genetic analysis information, I-Ching, specific knowledge based on philosophical analysis, iridology, vital signs, etc.) to generate customized compositions of supplements for each individual. A methodology, in which the customized compositions of supplements produced is applied to foods, beverages, and/or cosmetics, is also described.

### FIELD

The present invention relates to the fields of information and communication technology (ICT), Industry 4.0, and the functional foods industry.

### BACKGROUND OF THE INVENTION

Almost all consumers are aware of the recommended dietary allowance (RDA) for various essential nutrients, which is the average daily reference value to take for healthy individuals. These benchmarks (RDA) may be useful to help ordinary healthy individuals plan and evaluate nutrient intake. However, these benchmarks can hardly be applied for optimizing immunity protection and infection resistance for individuals not categorized as ordinary healthy.

Here are some examples (these are only a few out of many other examples): (1) Vitamin C. The vitamin C range of the RDA is approximately 40 to 110 mg. However, the appropriate amount of vitamin C supply required to optimize the function of cellular tissue in the body and improve immunity is 100-200 mg per day for individuals categorized as ordinary healthy. In the case of the treatment of infections, there is a need for much higher doses to compensate for the increased inflammatory responses and metabolic demands. (2) Vitamin E. For older adults (people who are not categorized as ordinary healthy), we know that higher doses of vitamin E (above the recommended levels for ordinary healthy people) could be required for functions such as helping restore T-cell functionality and helping improve vaccine efficacy. (3) Synthetic a-tocopherol is related to upper respiratory infection rates. (4) Vitamin B6 is required for lymphocyte proliferation.

This application does not intend on fixing and/or treating specific symptoms or diseases through the customized compositions of supplements. Instead, this invention smartly applies the analyzed and structured data via AI (Artificial Intelligence) and IoT devices into the foods and beverages (including cosmetic products). Further development related to the technologies and legal frameworks, which is required to communicate with the codes & regulations of the Food & Drug Administration, may cover the application of the treatments of certain symptoms and diseases based on the foundation of the concept of this invention in the future.

The basic concept of this invention is “Building AI algorithm(s)” to generate customized compositions of supplements (such as minerals and vitamins) for each individual through multiple methodologies (such as the types of constitutions of the human body in accordance with Oriental Medicine, I-Ching, blood types, Iridology, and etc.). This invention also consists of the method of applying customized compositions of supplements to the foods & beverages (including cosmetic products). The methodologies introduced in this invention provides customized compositions of supplements to the clients based on the analysis and classification of the body/health in the form of drinking beverages (especially bottled water), customized foods and/or cosmetics, which is free of chlorine fluoride and free from other harmful chemicals and heavy metals.

Our customized cosmetic products and foods & beverages (especially bottled mineral water that contains the customized composition of supplements) can also contain electrolytes. It is not intended to treat or cure any disease but instead helps users achieve a better lifestyle as well as benefit from the additives included in the customized composition of minerals.

A list of possible minerals that can be included (but is not limited to) is: calcium, potassium, magnesium, and selenium. This also includes almost all types of minerals such as the following: phosphorus, sulfur, chlorine calcium fluoride, manganese, iron, zinc, gallium, germanium, arsenic, selenium, rubidium, strontium, nitrate, niobium, molybdenum, rhodium, palladium, silver, cadmium, indium, tin, antimony, tellurium, iodine, cerium, neodymium, bismuth, polonium.

### BRIEF SUMMARY OF THE INVENTION

This invention involves engineering in relation to the classification and/or differentiation of the health-conditions of individuals to figure out applicable and beneficial customized compositions of supplements that can be added into beverages, foods, and cosmetic products.

This invention involves engineering in relation to the application of technologies to sense and/or monitor the health-conditions of individuals to figure out applicable and beneficial customized compositions of supplements that can be added into beverages, foods, and cosmetic product.

This invention involves engineering in relation to analytic data related to the vital signs of individuals retrieved from IoT devices (such as, but not limited to, the wearable smart watch) to generate information for the customized compositions of supplements to mix into foods, beverages, and cosmetic products.

This invention involves engineering in relation to the transmittal and utilization of the data collected via sensing and/or monitoring procedures to figure out applicable and beneficial customized solutions for the clients.

This invention involves engineering in relation to the application of the outcomes acquired via data-analytic and deep-learning procedures via various types of products used and/or consumed by clients.

### BRIEF DESCRIPTION OF THE SEVERAL VIEWS OF THE DRAWING

FIG. 1 shows the minerals, vitamins, and supplements that could be the components of drinking water and/or customized foods by adding to it. The steps are as follows.

FIG. 2 (a and b) shows the general flow that could be applied for monitoring in order to collect data, transmit data, and analyze data (including the methodology of sharing data between IoT devices (such as the wearable apple watch and the data receiving terminal).

FIG. 3 shows the flow of the transmittal of health-related data of individuals who are not registered in the system. The flow also shows the applicable methodology of the utilization of data for data analysis.

FIG. 4 shows the flow of the transmittal of health-related data of individuals who are registered in the system. The flow also shows the applicable methodology of the utilization of data for data analysis.

FIG. 5 shows the flow of the transmittal of health-related data of existing customers (does not record the session's information) in the system. The flow also shows the applicable methodology of the utilization of data for data analysis.

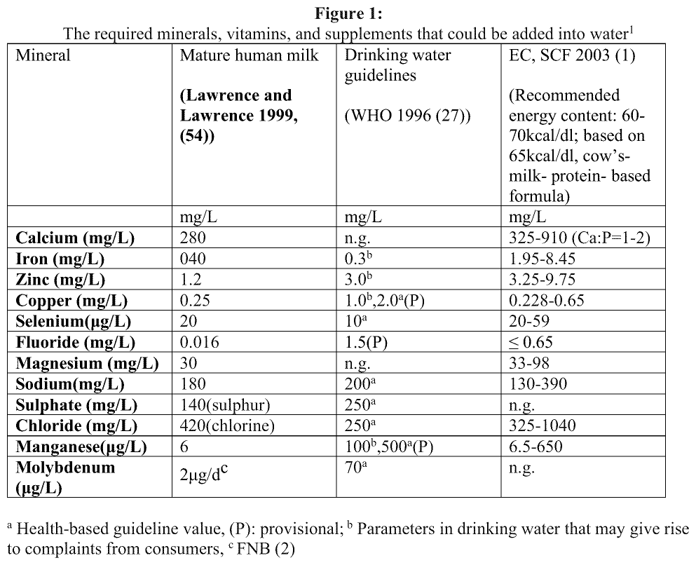

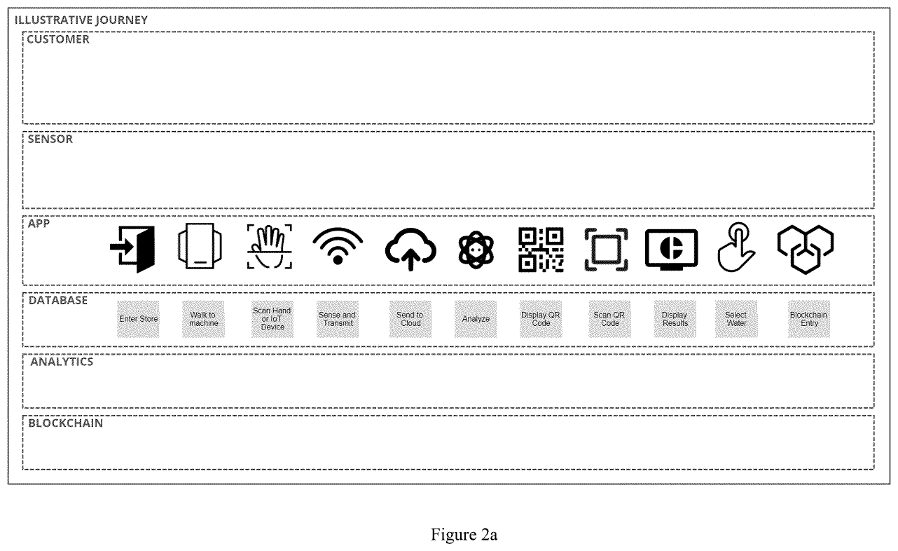

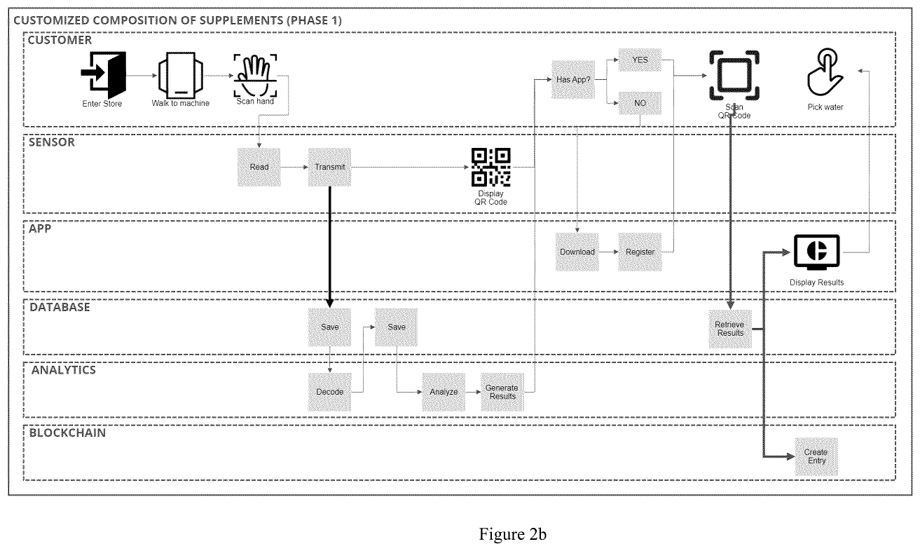

FIG. 6 shows the flow of the transmittal of health-related data of existing customers (record the session's information) in the system. The flow also shows the applicable methodology of the utilization of data for data analysis.
1. Collect health related data from monitoring devices (including, but not limited to, IoT based wearable smart watch) and transmit the data for further analytic procedures (such as structuring the data)
2. Sense and/or monitor the condition of the body/health via data collected from (not an exhaustive list) urine, hair, sweat, circulatory system, hormone secretion, bone density, and cell viability in one or more organs in the body.
3. Store the processed data (structured data/standardized data) into RAM (Random Access Memory)
4. Treat the structured data with the Algorithm engine (Business Rule Engine)
5. Apply the results of the data analytics to classify (and/or to grouping) the client based on various types of applicable methodologies (such as, but not limited to, the theory of ORIENTAL medicine, I-CHING, Blood Types, Analysis of basic Vital signs, HOMEOPATHIC MEDICINE, etc.)
6. The classifications of customized compositions of the supplements that can be added to foods, beverages, and cosmetic products can be decided based on the following: (a) The information of blood type of each individual, which can be obtained not via the physical blood test but via the statement provided by each individual. (b) The categorization of constitutions into 4 types, 8 types, 16 types, and 32 types according to the classifications addressed in the theory of Oriental Medicine and/or I-Ching. (c) The correlation between fundamental vital signs (such as, but not limited to: status of blood pressure, status of heartbeat, status of body temperature, status of respiration, sugar level in the blood) and health conditions.
7. Visualize the results through the monitor for the client (each individual). This monitor communicates with the client's IoT device(s) to retrieve the data required to formulate the customized composition of minerals for the client and it also has some example functions (this is not a complete list of functions. this is only a few out of many example functions): (a) Dispensing the foods, beverages, and/or cosmetic products with the added customized composition of minerals for the client. (b) Visualizing the results of the customized composition of minerals for the client to see. (c) Relaying the results to partnered vendors to add the customized composition of minerals into the foods, beverages, and/or cosmetic products for the client (for example, this monitor can be installed in a cafe and, upon receiving the data from the customer's IoT device(s), relay the information to the cafe so that the customized composition of minerals can be added to the customer's order)
8. Store the record for each individual's data/information so that it can be used for further machine learning and/or deep learning procedures

### DETAILED DESCRIPTION OF THE INVENTION

The method of making beverages (especially bottled water), foods and/or cosmetic products by adding the customized compositions of supplements (such as minerals, vitamins, and others) depends on the analysis of data related to the individual health status (body conditions. This status is collected by all sorts of devices, including (but not limited to) IoT devices, such as the wearable smart watch.

Any type of sensor (including, but not limited to, sensors based on Gallium Nitride, Magnetoencephalography Optically Pumped Magnetometers, Twin Core Fiber based sensor, and etc.) that generates health related raw data can be hired to be utilized by the AI-based algorithm to process data analytics.

Any kind of sensor (including, but not limited to, the Quantum sensor that exploits the laws of quantum mechanics and proves the measurements of physical quantities directly from values of fundamental constants of quantum physics) that can monitor the conditions of the health of the human body directly and/or indirectly can be hired/applied to work through at least the detection of the rate of heartbeat, blood pressure, body temperature, sugar level in the blood, urine, sweat, hair, circulatory system, hormone secretion, the detection of bone density, and the examination of the cells of the organs in the body. Monitoring sensors embedded into the form of wearable device (including, but not limited to, the wearable smart watch) shall also be utilized to generate health related data. This data that is being generated by these kinds of monitoring sensors via wearable IoT devices shall then be transmitted to our AI algorithm(s) to produce the information necessary for the customized compositions of supplements for each individual.

The composition of supplements (such as minerals, vitamins and others) could also be grouped together (hereinafter “GROUP”). (The GROUP includes CREATINE, PYRUVIC ACID and CARNITINE and depends on the analysis of data collected via sensing/monitoring procedures). The GROUP could also rely on theories for the classification(s) that are defined by ORIENTAL MEDICINE, Oriental philosophy (such as I-CHING), Blood types, DNA analysis, correlation between fundamental vital signs and health conditions and so on. At least one GROUP (customized composition of supplements) selected from many different groups of compositions could be added to foods, beverages, and cosmetic products.

Supplement such as Sodium chloride, calcium phosphorus, magnesium, sulfur, zinc iron, iodine selenium, copper, manganese, chromium, molybdenum and essential vitamins for the human body could also be considered to be added to the foods, beverages and/or cosmetic products in accordance with the data analysis for the individual's condition. The amount of minerals and vitamins added to the foods & beverages (such as, but not limited to, the bottled water) shall not go over the limitation recommended by the WHO (World Health Organization. In addition, the amount of supplements added shall be controlled safely in the frame of international standards applied to the manufacturer of foods & beverages and cosmetic products.

The purpose of this invention is to provide customized supplements to clients via beverages, foods, and/or cosmetic products based on the analysis of data related to the conditions of the body/health of each individual using/applying any type of device (especially IoT based devices that use any type of sensors) based on the theories. These theories and/or scientific facts include, but are not limited to, Oriental Medicine, Western Medicine, Blood Types, correlation between fundamental vital signs and human health, and any theory/method for the classification of the different types of constitutions of human bodies.

Calcium, phosphorus, magnesium, sulfur, zinc iron, iodine, selenium, copper, manganese, chrome, molybdenum and other supplements (including special vitamins) could also be considered and added according to the analysis of the data that deals with the condition of the body/health of each individual.

The amount of minerals, vitamins, and other supplements added to the drinking water could be delivered via automatic dispensing or automatic drinking water dispensers.

Automatic drinking water dispensers are strictly controlled by a data processor that controls the safe criteria for producing personalized drinking water.

After monitoring health conditions, the data obtained shall be structured for utilization in the algorithms used to identify individualized combinations of supplements that may be beneficial to individual health. The output, which is the result from the algorithm, shall generate a customized combination of supplements (individualized minerals) that needs to be applied to the beverages, foods, and cosmetics.

All data generated by health-related sensors are linked to the databases on external networks via cloud and are being utilized to calculate and decide the customized composition of supplements (necessary minerals, vitamins, and others) for each individual. The AI based algorithm matches the composition of mineral ingredients, which will be added to foods, beverages, and cosmetic products. All the data collected through entire tasks mentioned above should be structured and stored for further tasks, especially for the optimum maintenance of each individual via machine learning.

The best mode of carrying out the invention: As mentioned, the methodology utilizes the data being generated by the IoT devices (such as, but not limited to, the wearable smart watch) to generate information for the customized compositions of supplements to add to the foods, beverages, and cosmetic products for each individual.

The invention will do the following:
1. Collect health-related data from various type of sensors (including, but not limited to, the one which is embedded in the wearable smart watch)
2. Apply the collected data for further data analysis in order to generate information for the customized/individualized supplements
3. Add the selected composition of supplements to the categorized foods, beverages, and/or cosmetic products.

Steps 1-3 under paragraphs 4-9 is explained using the drawings provided below.

### REFERENCES

SCF, Scientific Committee on Food. Report of the Scientific Committee on Food on the Revision of Essential Requirements of Infant Formulae and Follow-on Formulae. SCF/CS/NUT/IF/65, 2003.
FNB, Food and Nutrition Board, Institute of Medicine. Dietary Reference Intakes: Vitamin A, Vitamin K, Arsenic, Boron, Chromium, Copper, Iodine, Iron, Manganese, Molybdenum, Nickel, Silicon, Vanadium and Zinc. Washington DC: National Academy Press, 2001.

1. The generation of information for the customized composition of supplements (such as minerals, water, etc.) for each individual.

2. The application of any type of sensors (including, but not limited to, Quantum technology applied sensors and sensors that work with IoT devices) for the collection of health-related data to generate information for the customized composition of supplements for each individual.

3. The application according to claim 2, wherein sensors collect data from urine, hair, sweat, blood circulation, hormone secretion, bone density, etc.

4. The utilization of health-related data being generated by the IoT devices (such as, but not limited to, the wearable smart watch) to generate information according to claim 1.

5. The transmittal (pushing) of the collected health related data from the IoT devices (such as, but not limited to, the wearable smart watch) to the cloud for data analytics to generate information according to claim 1.

6. The transmittal of the result of data analytics from the AI-based algorithm to the terminal for the clients (each individual) to read.

7. The application of the information generated according to claim 1 to beverages, foods, and/or cosmetic products for each individual upon their decision.

8. The generation of information for the customized composition of supplements based on the health-related data collected from each individual by applying the information classified based on the analysis of information.

9. The generation of information according to claim 8, wherein the analysis of information is of the blood types of the human.

10. The generation of information according to claim 8, wherein the analysis of information is of DNA information of each individual.

11. The generation of information according to claim 8, wherein the analysis of information is of vital signs (such as, but not limited to, blood pressure, oxygen resolved in the blood, heartbeat, body temperature, sugar level in the blood, and etc.)

12. The generation of information according to claim 8, wherein the analysis of information is from iridology.

13. The generation of information according to claim 8, wherein the analysis of information is from the classification of the human body and human health by ORIENTAL Medicine.

14. The generation of information according to claim 8, wherein the analysis of information is from the classification of the constitutions of the human body according to the oriental philosophy (such as I-CHING).

15. The generation of information according to claim 8, wherein the analysis of information is from other traditional methods (such as, but not limited to, traditional Indian alternative medicine) that differentiates the constitutions of human bodies.

16. The multiple methodologies used for the classification of the individualized components of supplements (including, but not limited to, required minerals, vitamins, and other supplements that could be added into food, beverage, and/or cosmetic products) based on the analysis of data collected via sensing/monitoring procedures for individuals.

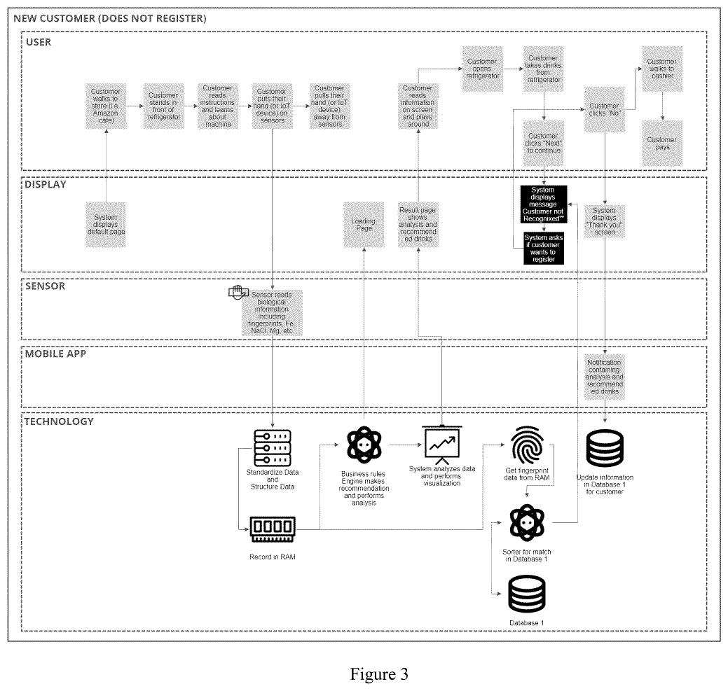

17. The methodologies according to claim 16, wherein the individuals are not registered in the system (FIG. 3).

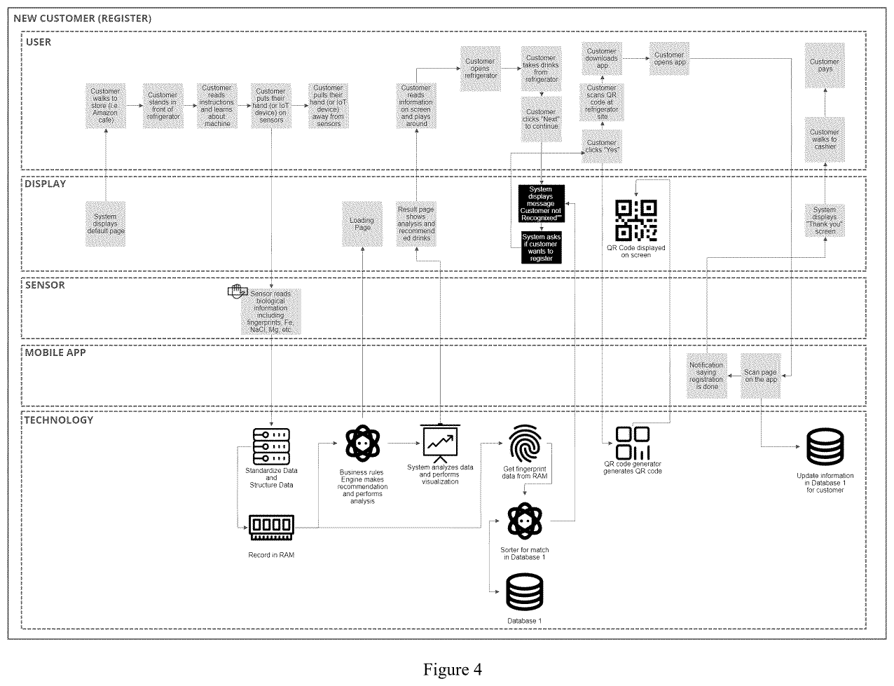

18. The methodologies according to claim 16, wherein the individuals are registered in the system (FIG. 4).

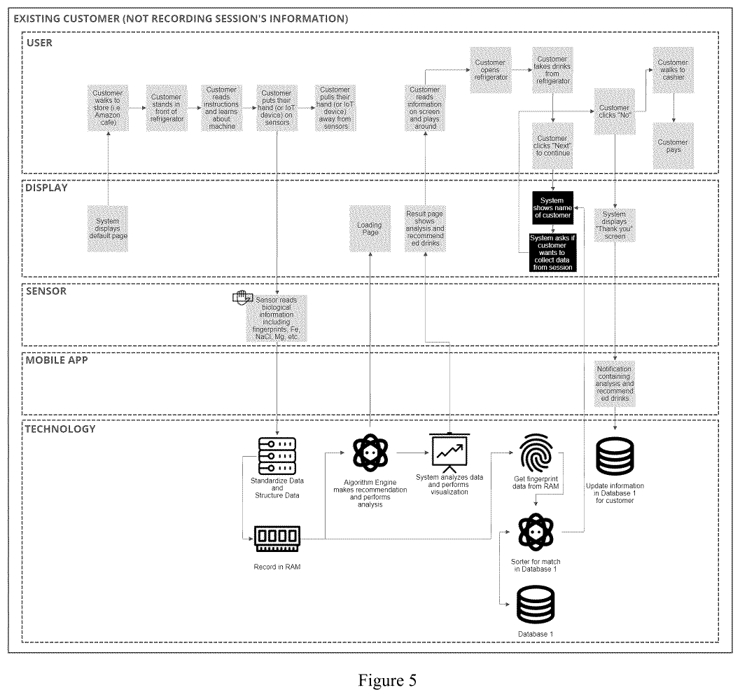

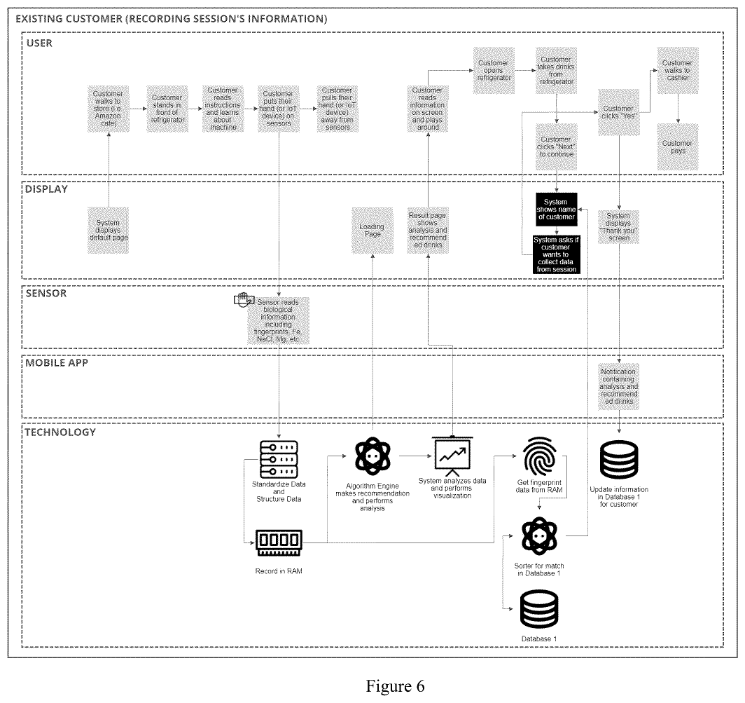

19. The methodologies according to claim 16, wherein the individuals are existing customers without recording the session's information (FIG. 5).

20. The methodologies according to claim 16, wherein the individuals are existing customers with recording of the session's information (FIG. 6).

21. The application of various theories and/or scientific information (such as, but not limited to, Oriental medicine, Oriental philosophy ‘I-Ching’, specific characteristics of health condition based on blood type, analyzed information for DNA, correlation between condition of health and basic vital signs, and etc.) to classify the GROUPs that cover multiple numbers of the customized compositions according to claim 1.

22. The selection of the ingredients for the customized compositions according to claim 1 to not only from the supplements such as calcium, potassium, magnesium, selenium, but also from almost all types of minerals (including, but not limited to, phosphorus, sulfur, chlorine calcium fluoride, manganese, iron, zinc, gallium, germanium, arsenic, selenium, rubidium, strontium, nitrate, niobium, molybdenum, rhodium, palladium, silver, cadmium, indium, tin, antimony, tellurium, iodine, cerium, neodymium, bismuth, polonium).

23. Defining the GROUP: The composition of supplements according to claim 1 (such as, but not limited to, minerals, vitamins and others) could also be grouped (The GROUP includes CREATINE, PYRUVIC ACID and CARNITINE and depends on the analysis of data collected via sensing/monitoring procedures as well as rely on theories for the classification(s) that are defined by the ORIENTAL MEDICINE, Oriental philosophy (such as the I-CHING), Blood types, DNA analysis, correlation between fundamental vital signs and health conditions, and so on.

24. At least one GROUP (customized composition of supplements) selected from many different groups of compositions could be decided by the data analytics and the selected compositions of supplements that are added to foods, beverages, and cosmetic products.

25. The application of the dispensing system involves the amount of minerals, vitamins and other supplements added to the beverage, food, and/or cosmetic products will be provided through the automatic dispensing system (such as, but not limited to, in the manner of an automatic drinking water dispenser).
